(19) United States
(12) Patent Application Publication
(10) Pub. No.: US 2025/0000454 A1
(43) Pub. Date: Jan. 2, 2025

(54) WEARABLE OPIOID OVERDOSE DETECTION AND ALERT APPARATUS

(71) Applicant: Kyle BERMAN, Birmingham, AL (US)

(72) Inventor: Kyle BERMAN, Birmingham, AL (US)

(21) Appl. No.: 18/738,855

(22) Filed: Jun. 10, 2024

Related U.S. Application Data
(60) Provisional application No. 63/523,404, filed on Jun. 27, 2023.

(51) Int. Cl. A61B 5/00; A61B 5/145

(52) U.S. Cl. CPC A61B 5/4845; A61B 5/14542; A61B 5/681; A61B 5/7282; A61B 5/746; A61B 5/747; A61B 2560/0257

(57) ABSTRACT

System involved with but not limited to an apparatus including a wearable electronic device configured to detect an opioid overdose and initiate transmission of an emergency notification to emergency services or an emergency contact. A wearable opioid detection device may detect an opioid overdose by measuring a drop in user's blood oxygen level and initiate an alarm notifying the user of decreased blood oxygen concentration. The apparatus and method of use thereof is capable of triggering an alert and communicating the wearer's location to emergency services or contacts when the user fails to cancel an alarm generated when the wearer's oxygen saturation falls below their normal saturation trend or particular fixed value.

### RELATED APPLICATIONS

This non-provisional application claims the benefit of U.S. Provisional Application No. 63/523,404, filed Jun. 27, 2023. U.S. Provisional Application No. 63/523,404 is hereby incorporated by reference in its entirety.

### TECHNICAL FIELD

Embodiments of the present disclosure relate generally to wearable health monitoring devices. More specifically, aspects of the present disclosure relate to opioid overdose detection methods and systems utilized in conjunction with a wearable device configured to measure decreases in the saturation of oxygen in the blood of the wearer. Further aspects of the present disclosure include methods of transmitting an emergency signal or message, which may be managed through the use of components capable of directly notifying emergency services or contacts or indirectly doing so by utilizing another device, such as a smartphone, as well as detecting and transmitting the geographic and altitude location of the wearer to said emergency services and contracts.

### BACKGROUND

Opioid overdoses have become a critical public health issue, with an alarming increase in opioid-related deaths worldwide. Opioids, including prescription painkillers such as fentanyl and illicit substances like heroin, pose a significant risk due to their potential to cause respiratory depression, leading to fatal consequences if left untreated. The Centers for Disease Control and Prevention estimate that over 100,000 people died of opioid overdose in the United States alone in 2022. Prompt intervention is crucial in saving lives during opioid overdose incidents, but timely detection can be challenging, particularly when the individual is alone or lacks immediate access to emergency medical services.

Existing approaches to opioid overdose detection primarily rely on bystander intervention or the presence of traditional monitoring devices, such as electrocardiograms, which may not be readily available or suitable for continuous monitoring in everyday situations. These methods often suffer from limitations such as reliance on external monitoring devices, lack of real-time alerts, inability to detect overdoses when the individual is unresponsive or unable to notify others of distress, or when the individual is alone.

Opioid overuse can induce respiratory depression by causing a decrease in involuntary respiratory rate, which in turn causes a decrease in oxygen saturation in the blood of an individual. To address the challenge of rapidly detecting an opioid overdose and quickly obtaining emergency assistance, a wearable detection device capable of accurately and quickly identifying opioid overdoses and transmitting an alert and the wearer's location may facilitate timely, potentially life-saving intervention.

A wearable opioid overdose detection device may be unobtrusive and discreet both to decrease any discomfort associated with consistent wear, as well as prevent potential stigma associated with opioid use. Stigma and judgment associated with any wearable article indicating opioid use could cause users to hesitate to wear such devices, seek help when needed, or disclose their opioid use or associated health condition to others. The present disclosure may overcome this challenge through simplicity of design and wear, as well as being discreet and unobtrusive.

### SUMMARY

It is an object, feature, and/or advantage of the present disclosure to provide a wearable opioid overdose detection and emergency contact alert apparatus and methods of use thereof that overcome deficiencies in the prior art. In accordance with one exemplary aspect, an exemplary embodiment of a wearable opioid overdose detection device may comprise a) one or a plurality of pulse oximeters configured to measure the saturation of oxygen (SpO₂) in the wearer's blood, b) a GPS unit configured to detect the location of the wearer, c) a communications module capable of transmitting a distress signal and the location of the wearer, d) a power source, such as a battery, e) an alert notification system such as a vibration motor and/or speaker, f) an analog user input button, and g) a computerized control module. This component list is not intended to be limiting as optional configurations may omit one or more of the preceding list of components. A wearable opioid overdose detection device may optionally feature an electronic barometer in order to approximate the wearer's relative altitude and consequently his or her floor location within a given building and/or an accelerometer to detect lack of movement indicating a preferred opportunity to obtain SpO₂ readings, which are more accurate when the wearer is still. These components may be disposed in an unobtrusive band worn on the user's wrist. The pulse oximeter component of the system is configured to monitor and analyze the user's blood oxygen saturation. A known biological effect of an opioid overdose is respiratory depression, which may manifest in a large or rapid decrease in blood oxygen saturation. Upon detecting a significantly large or rapid decrease in blood oxygen saturation, the device triggers the activation of an alarm, which may be signaled by a vibration motor. The vibration motor may generate tactile vibrations to alert the user of the abnormal condition and of a pending emergency signal. If the user fails to deactivate the alert within a predefined period of time, the system may initiate the transmission of an emergency message either directly via transmitter disposed in the wearable opioid overdose detection device or by utilizing the wearer's smartphone via an associated software application. This emergency message may be sent to preconfigured emergency contacts, such as friends, family members, or healthcare providers, or directly to emergency medical services (EMS). An exemplary emergency message includes the wearer's location, as measured by said GPS module, and the approximate floor of the individual as corresponding with the altitude as measured by the electronic barometer and cross-referenced with weather data to account for atmospheric pressure variations. The message may be transmitted through various communication channels, such as SMS, email, or wireless data networks.

### BRIEF DESCRIPTION OF THE DRAWINGS

The accompanying drawings, which are incorporated in, and which constitute a part of this specification, illustrate exemplary constructions and procedures in accordance with the present disclosure and, together with the general description of the disclosure given above and the detailed description set forth below, serve to explain the principles of the disclosure wherein:

FIG. 1 illustrates a perspective view of the exterior of an exemplary wearable opioid overdose detection and alert apparatus;

FIG. 2 illustrates a component map of an exemplary configuration of an exemplary wearable opioid overdose detection and alert apparatus;

FIG. 3 illustrates a lateral view of the exterior of an exemplary wearable opioid overdose detection and alert apparatus;

FIG. 4 is an exemplary flow chart illustrating a calibration command and control logic in the event the device detects a drop in blood oxygenation level indicating a potential opioid overdose; and

FIG. 5 is an exemplary flow chart illustrating the use of an accelerometer to detect preferred opportunities to take a wearer's measurement (SpO₂ readings are more accurate when the wearer is still) and to reduce the frequency of measurements when the wearer is asleep to preserve battery life.

While constructions consistent with the present disclosure have been illustrated and generally described above and will hereinafter be described in connection with certain potentially preferred embodiments and practices, it is to be understood that in no event is the disclosure limited to such illustrated and described embodiments and practices. On the contrary, it is intended that the present disclosure shall extend to all alternatives and modifications as may embrace the general principles of this disclosure within the full and true spirit and scope thereof. Also, it is to be understood that the phraseology and terminology used herein are for purposes of description only and should not be regarded as limiting. The use herein of terms such as “including” and “comprising” and variations thereof is meant to encompass the items listed thereafter and equivalents thereof as well as additional items and equivalents thereof.

### DETAILED DESCRIPTION

It is an object, feature, and/or advantage of the present disclosure to provide a wearable opioid overdose detection and emergency contact alert apparatus and methods of use thereof that overcome deficiencies in the prior art. More specifically, the present disclosure provides a wearable opioid overdose detection device capable of detecting significant and rapid decreases in blood oxygen saturation (SpO₂) that are associated with an opioid overdose. When an opioid overdose is detected by a rapid or large decrease in the wearer's SpO₂, the device may vibrate for a set period of time to offer the user an opportunity to cancel the emergency alert. When the user fails to cancel the alert, the device may transmit an emergency signal, along with critical information such as the wearer's condition and location, to preconfigured emergency contacts, which may be set as friends, family members, or emergency medical services. “SpO₂” and “blood oxygen saturation” may be used interchangeably throughout the disclosure.

In accordance with one exemplary aspect, a wearable opioid overdose detection and emergency contact alert apparatus is provided having one or a plurality of pulse oximeters configured to detect the oxygen saturation of the wearer's blood disposed in a wearable wrist band. Multiple pulse oximeters provide a more consistently accurate reading of the wearer's oxygen saturation by overcoming factors that affect the accuracy of a pulse oximeter reading, such as poor circulation, skin thickness, and skin pigmentation, which may vary with respect to different locations on a wearer's wrist. In an exemplary configuration of a wearable opioid overdose detection device, pulse oximeters may be disposed on opposing sides of the wristband and may be capable of reading oxygen saturation from the dorsal and ventral sides of the wrist. In such a configuration, the two pulse oximeters may take a reading from areas of the wrist with differing degrees of pigmentation or skin thickness. In addition, when movement of the wrist or other conditions interfere with the reading of one pulse oximeter, the other may be able to continue detection of the wearer's SpO₂. An exemplary control module provided in electronic communication with one or a plurality of pulse oximeters monitors the wearer's SpO₂. In configurations comprising a plurality of pulse oximeters, an exemplary control module calculates the wearer's SpO₂ in real time by averaging the detected values, or in the event of a detection error by one or more pulse oximeters, uses the remaining reading or an average of the remaining readings to provide continuous detection of the wearer's SpO₂.

A wearable opioid overdose detection device may be initially calibrated to the individual wearer's typical SpO₂. Those of skill in the art will appreciate that normal oxygen saturation is typically between 95-100%, but individual circumstances may cause a lower baseline blood oxygen saturation. For example, a person with chronic obstructive pulmonary disease may trend between 91-94%, while another person with respiratory illness or complications from COVID-19 could trend between 87-92%. The individual wearer's SpO₂ baseline may be calculated upon initial calibration of the device, wherein the device calculates the wearer's baseline oxygen saturation over a period of at least 60 minutes. Other modes of determining baseline may be provided, including the user inputting their own baseline via smartphone application or other electronic means wirelessly connected to an exemplary control module. In an exemplary, non-limiting embodiment, at any such time the wearer's average SpO₂ over a 180 second period drops by a value equal to or more than 3% from the wearer's SpO₂ baseline, the disclosed device detects an overdose event and initiates its overdose control logic. This manner of detecting an overdose is exemplary and is not intended to be limiting. For example, an overdose may also be detected by a drop in the wearer's SpO₂ by a value equal to or greater than 5% for any period of time, rather than over a 180 second period of time. Note that throughout this disclosure a percentage refers to oxygen saturation as a measure of how much hemoglobin is currently bound to oxygen compared to how much hemoglobin remains unbound, not a percentage as expressed in a change from a baseline number to a subsequent number.

When an overdose event signified by a significant decrease in blood oxygen saturation (SpO₂) is detected, an exemplary alert logic may be initiated. In an exemplary embodiment of alert logic, the disclosed device activates an alarm to alert the wearer of the overdose event and prompt immediate action by the wearer. The alarm may be generated by the vibration motor and/or a sound emanating from a built-in speaker provided in electronic communication with an exemplary control module, emitting a distinctive sound and/or tactile vibrations for a predetermined duration, such as 45 seconds. During this period, the wearer may cancel the alarm and address the abnormal condition. In an exemplary embodiment of the disclosed device, the user may cancel the alarm by pressing an analog button disposed on the wearable device. Other cancellation commands may also be programmed to minimize the possibility of an accidental cancellation. For example, a cancellation command may require the user to press a button multiple times within a certain time period, or hold down the button for a certain amount of time. If the wearer fails to cancel the alarm and the blood oxygen saturation does not return to the calculated baseline, the alarm process will repeat over a set period of time, for example, every 5 minutes. This repetition ensures that the wearer receives consistent and persistent reminders to address the potential opioid overdose. In an exemplary embodiment, if the wearer removes the device during a detected overdose event or fails to cancel the alarm within the specified time, the device may initiate a distress call or signal. Removal of the device may be detected when the pulse oximeter is unable to detect any oxygen saturation. In an alternative embodiment, a distress call or signal may be initiated following detection of an overdose event without an alert logic intermediate step.

The computerized control module includes a processor and a memory that communicate with each other, and is provided in electronic communication with all electronic components comprising a wearable opioid overdose detection and alarm device. Memory can include various components (e.g., machine-readable media) including, but not limited to, a random-access memory component, a read-only component, and any combinations thereof. Memory can also include (e.g., stored on one or more machine-readable media) instructions (e.g., software) embodying any one or more of the aspects and/or methodologies of the present disclosure. In another example, memory can further include any number of program modules including, but not limited to, an operating system, one or more application programs, other program modules, program data, and any combinations thereof. A user can also input commands and/or other information to an exemplary control module via another device with direct user interface (e.g., a smartphone, personal computer, etc.) via an exemplary data port, such as USB-C, provided in electronic communication with the computerized control module. A network interface device, such as a communications module discussed below, can be utilized for connecting the control module to one or more of a variety of networks, such as a phone or Wi-Fi network, and one or more remote devices connected thereto. It should be noted that the Wi-Fi technology is the industry name for wireless local area network communication technology related to the IEEE 802.11 family of wireless networking standards by Wi-Fi Alliance. Examples of a network include, but are not limited to, a wide area network (e.g., the Internet, an enterprise network), a local area network (e.g., a network associated with an office, a building, a campus, or other relatively small geographic space), a telephone network, a data network associated with a telephone/voice provider (e.g., a mobile communications provider data and/or voice network), a direct connection between two computing devices, and any combinations thereof. Information (e.g., data, instructions, etc.) can be communicated to and/or from the computerized control module via an exemplary communications module.

The communications module of an exemplary wearable opioid overdose detection device may be configured to transmit a distress call or signal to notify emergency medical services or an emergency point of contract designated by the wearer and receive communications from other electronic devices. An exemplary communications module may comprise a radio frequency (RF) module capable of causing an antenna structure to radiate electromagnetic energy in one or more frequency bands, including RF bands associated with a wireless area network (WAN), a wireless local area network (WLAN), a global positioning system (GPS), or the like. An exemplary communications module may also comprise an RF receiver capable of receiving radio signals in the aforementioned frequency bands. The communications module, in combination with the control module and power source, allows the device to send and receive data communications (such as communications for text messages, emergency calls, smartphone applications associated with the wearable opioid overdose detection and alert device, etc.) with a wireless communication system. The communications module may provide network connectivity using various types of digital mobile network technology including, for example, LTE, LTE advanced (4G), WLAN (e.g., Wi-Fi network), Bluetooth, etc.

The distress call or signal comprises a message containing information regarding the overdose incident. This information may include the confirmation of an overdose occurrence, the location of the wearer, and the wearer's current blood oxygen saturation (SpO₂) level. An exemplary message may be transmitted in multiple formats, such as SMS text message or a phone call utilizing pre-recorded or machine-generated verbiage, and may be transmitted directly by the wearable opioid overdose detection device itself. Alternatively, the device may leverage the wearer's smartphone, connected via Bluetooth or another wireless communication mode, to transmit the distress call or signal through the wearer's smartphone via a dedicated smartphone application. An exemplary distress call or signal is capable of being directed to emergency medical services (EMS) or an emergency point of contact designated by the wearer, such as a friend, family member, or healthcare provider. An exemplary opioid detection device may also transmit an emergency message or signal to a dedicated dispatch center.

An exemplary wearable opioid overdose detection device may utilize the features of a wearer's smartphone via an associated smartphone application, providing wearers with additional control and customization options. A dedicated smartphone application may serve as an interface for the wearer to input their personalized settings, preferences, and emergency contact information. The wearer may also utilize the application to set their individual SpO₂ baseline, which serves as a reference point for detecting significant drops in blood oxygen saturation. An exemplary smartphone application also allows wearers to designate preferred emergency contacts to be contacted when an overdose event is detected by the device. An exemplary smartphone application may also provide flexibility in adjusting the duration of the alarm triggered by the device and/or the mode of inputting an alarm cancellation command. An exemplary smartphone application may also allow the overdose detection device to utilize the communication capabilities of the wearer's smartphone to facilitate the transmission of the distress signal or message, or entirely outsource the function of transmitting the emergency signal or message to the smartphone. In this manner, an exemplary embodiment of a wearable opioid detection device need not include components capable of transmitting SMS or voice calls over applicable networks when paired with a smartphone capable of performing these functions.

An exemplary embodiment of a wearable opioid overdose detection device may comprise a location module to accurately determine the wearer's location during a detected overdose event. A location module generally determines a current geolocation of the device and may receive and process radio frequency (RF) signals from a multi-constellation global navigation satellite system (GNSS) such as the global positioning system (GPS), the GLONASS system, the Galileo system, or the like. The location module, in combination with a computerized control module may include satellite navigation receivers, processors, controllers, other computing devices, or combinations thereof, and memory. The location module may process a location electronic signal received or communicated from the location determining antenna, which receives one or more location wireless signals from one or more satellites of the GNSS. The location wireless signal includes data from which geographic information, such as the current geolocation of the device, is determined by the location module. The current geolocation may include coordinates, such as the latitude and longitude, of the current location of the device. In an alternative embodiment, a wearable opioid overdose detection device may utilize the GPS module of a smartphone in which an associated smartphone application is installed to determine the wearer's location during a detected overdose event.

An exemplary embodiment of a wearable opioid overdose detection device may comprise a barometer configured to detect the altitude of the wearer. It is known by those practicing in the art that altitude information detected by a barometer may be cross-referenced against known elevation values of the wearer's location to determine the wearer's distance from the ground when such information accounts for local weather information to account for temporary variations in pressure and other atmospheric conditions. Using this information, an approximate plane location on an individual floor of a building may be provided with location information during a detected overdose event. In an alternative embodiment, a wearable opioid overdose detection device may utilize the barometer module of a smartphone in which an associated smartphone application is installed to determine the wearer's indoor floor location during a detected overdose event.

An exemplary embodiment of a wearable opioid overdose detection device may comprise an accelerometer configured to detect the movement of the wearer. When an accelerometer detects a wearer's lack of movement, the device may take the wearer's SpO₂ measurement. Those skilled in the art will appreciate that SpO₂ readings obtained by pulse oximeters are typically more accurate when the user is still rather than moving. In this manner, the accuracy of the device's measurements may be improved. In an exemplary embodiment of this feature, a wearable opioid overdose detection device may be programmed to take an SpO₂ measurement after the wearer has been still for a set period of time, such as 30 seconds, 1 minute, or 2 minutes, and continue to take readings at set intervals as the wearer remains still, such as every 3 minutes. If no movement is detected for protracted durations and SpO₂ levels are measured in the wearer's baseline range, a wearable opioid overdose detection device may be programmed to increase the duration of time between SpO₂ measurements to save battery power because the individual is likely sleeping. For example, if the accelerometer detects no movement for a period of 15 minutes or more, a wearable opioid overdose detection device may shift from taking SpO₂ readings every 3 minutes to every 20 minutes until the accelerometer again detects motion indicating the wearer is awake.

An exemplary wearable opioid overdose detection device may comprise a band made of microfilament material, or other materials suitable for a wristband. An exemplary microfilament band offers flexibility and breathability, ensuring long-term comfort without causing irritation, as well as elasticity, helping to ensure that pulse oximeters disposed in the band are maintained in a position close to the skin in which they may continuously and accurately the wearer's oxygen saturation. Consideration of comfort is critical given the fact that the device must be worn long-term in order to achieve its desired effect. The band of an exemplary opioid detection device may be offered in a variety of designs, patterns, and colors, allowing wearers to choose a style that aligns with their personal preferences. Such an exemplary design improves the device's capacity for discreet and inconspicuous use, minimizing any potential stigma associated with opioid use or overdose. By offering different designs, the wearable device can blend seamlessly with everyday attire, further reducing any potential social or psychological barriers that may discourage users from wearing the device. Electronic components of the device, including the pulse oximeter, GPS module, transmitter, control module, barometer, accelerometer, and other associated components, may be housed within one or a plurality of waterproof or water-resistant metal enclosures. Such enclosures may provide protection to the electronic components from moisture, dust, and other environmental factors. The electronic components within each enclosure may be provided in electronic communication with each other and with components housed in other enclosures, with wired elements optionally integrated into the band itself. It should be noted that while the use of waterproof or water-resistant metal enclosures is described herein, other suitable containers or casings made of different materials may also be employed to protect the electronic components.

A wearable opioid overdose detection device comprises a power source. In one embodiment, a wearable opioid overdose detection device incorporates a rechargeable battery, which may be easily recharged using a compatible charging unit, such as a USB-C cable connected to a power source or a wireless charging pad. In an alternative embodiment, a wearable opioid overdose detection device may utilize a replaceable battery, providing flexibility in terms of power management. A replaceable battery may allow a wearer to simply swap out the depleted battery with a fully charged one, eliminating the need for waiting for recharging cycles. Embodiments of the disclosed device may support standard battery types, such as coin cell batteries or rechargeable battery packs, making it readily adaptable to various power requirements.

Many components comprising a wearable opioid overdose detection device and their corresponding functions may be found in modern smartphones, such as GPS location services, wireless network connectivity, motion detection, etc. Some embodiments of the device may outsource one or more of the functionalities disclosed herein to a smartphone provided in wireless communication (such as via Bluetooth) with the device, whether or not the corresponding component is included within the device. For example, an embodiment of a wearable opioid overdose detection device may not include a location module, and may rely upon the location capability of a smartphone provided in communication therewith for location functionality. Alternatively, a wearable opioid overdose detection device may have standalone location services capability, but may utilize a smartphone's location services to save battery.

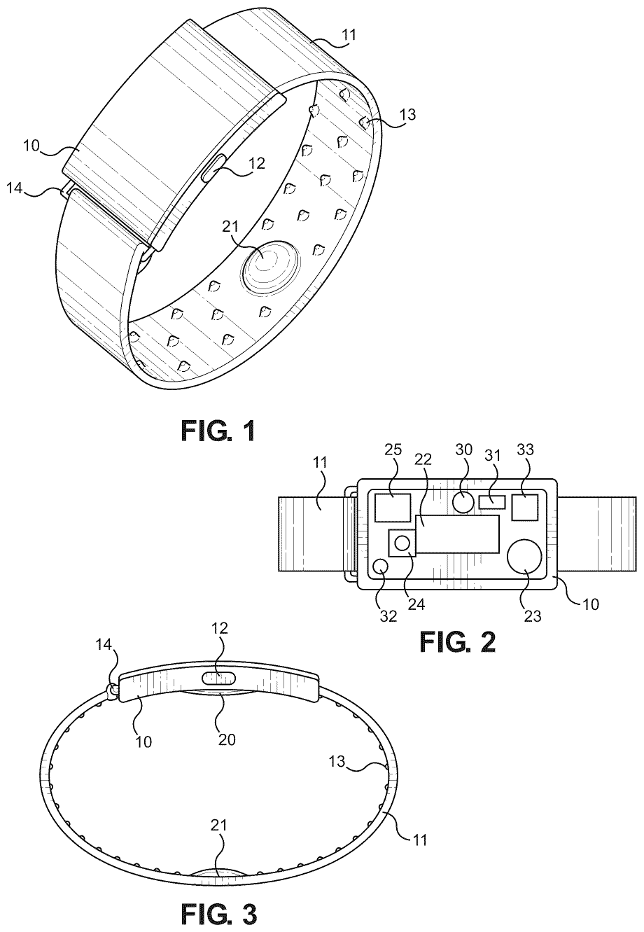

Referring now to the drawing wherein like numerals refer to like parts in the various views, FIG. 1 illustrates a perspective view of the exterior of an exemplary wearable opioid overdose detection and alert apparatus. The illustrated exemplary wearable opioid detection and alert apparatus includes an electronic components housing 10, a wristband 11 (which may be optionally adjustable via the use of a lug 14), an analog button 12 capable of communicating a signal to the computerized control module, projecting studs 13 to enhance the grip of the wearable device and reduces the movement of an SpO₂ sensor 21, thereby improving SpO₂ measurement accuracy. An antenna (not shown) may be embedded into the housing 10 and/or wristband 11.

FIG. 2 illustrates a component map of an exemplary configuration of an exemplary wearable opioid overdose detection and alert apparatus. An electronic component housing 10, which may be waterproof or water resistant, attached to an adjustable wristband 11 encloses and protects electronic components of the device. In this exemplary embodiment, power is supplied by a battery 23 provided in electronic communication with all electronic components of the device. A control module 22 is provided in electronic communication with all electronic components of the device and sends and receives electronic communications from internal electronic components, including but not limited to a communications module 24, a GPS module 25, an electronic barometer 30, an accelerometer 31, and an alarm system comprising a sound speaker 32 and vibration module 33.

FIG. 3 illustrates a lateral view of the exterior of an exemplary wearable opioid overdose detection and alert apparatus showing SpO₂ sensors oriented to measure the wearers blood oxygen saturation in two locations. A first SpO₂ sensor 20 disposed on the bottom exterior of the electronic components housing 10 may provide an SpO₂ measurement on the dorsal side of the wearer's wrist, while a second SpO₂ sensor 21 disposed on the surface of the wristband 11 opposite a first SpO₂ sensor 20 may provide an SpO₂ measurement on the ventral side of the wearer's wrist. This orientation is not intended to represent the best mode of use as a wearer may wear the device “upside down” (i.e. with the first SpO₂ sensor is located on the ventral side of the wearer's wrist and the second SpO₂ sensor is located on the dorsal side of the wearer's wrist) and obtain similar, if not the same results. A second SpO₂ sensor 21 is provided in electronic communication with a control module 22 via a wire embedded within the wristband 11. This wire (not shown) may be disposed within the wristband 11 in a “zigzag” orientation wherein the wire is shaped into a series of alternating turns at acute angles. This orientation offers the wire some degree of flexibility or “stretch,” wherein the effective length of wire may be extended somewhat as the ends are pulled apart and the “zigzag” angles increase in degree. This lateral view also shows an exemplary analog button 12 and optional grip studs 13.

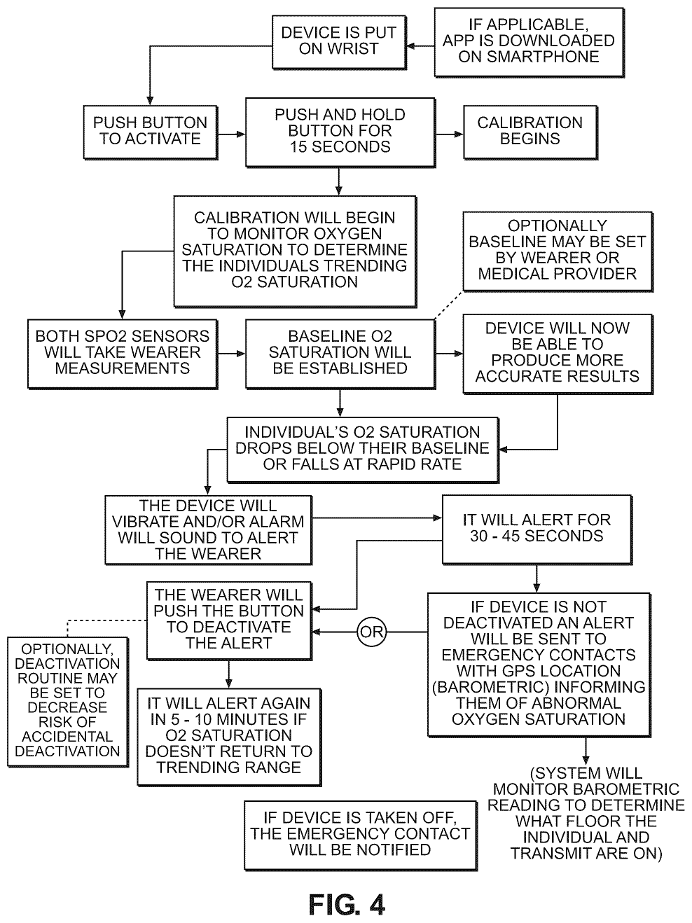

FIG. 4 is an exemplary flow chart illustrating a calibration command and control logic in the event the device detects a drop in blood oxygenation level indicating a potential opioid overdose. In this exemplary calibration and control logic, the user places the device on his or her wrist (and optionally downloads an associated smartphone application) and presses the analog button to turn on the device. The wearer may initiate calibration by pressing and holding the button for an extended period of time, in this example, 15 seconds. Once the device has calibrated and determined the user's baseline SpO₂ (or the user otherwise inputs the baseline), it monitors the wearer's SpO₂ at regular or other intervals. In an exemplary embodiment comprising two SpO₂ sensors, SpO₂ is determined by an average of the wearer's SpO₂ as measured by both sensors, or one SpO₂ sensor alone if one measurement is not able to be taken or is clearly erroneous by virtue of being far outside an expected value. If the wearer's SpO₂ drops below their baseline for an extended period of time or quickly falls a larger margin below baseline, the device's exemplary alert logic may begin. The device will vibrate and/or issue an alarm sound for a period of 30-45 seconds until the wearer deactivates the alert using the analog button. The deactivation signal may require the user to hold down the button for an extended period or press the button a certain number of times to reduce the risk of an accidental deactivation. If the device is not deactivated, it will send an alert communication to predetermined emergency contacts, which may include indication of a health emergency, location, and approximate floor level. The device may also be configured to alert a predesignated emergency contact if it is removed from the wearer's wrist.

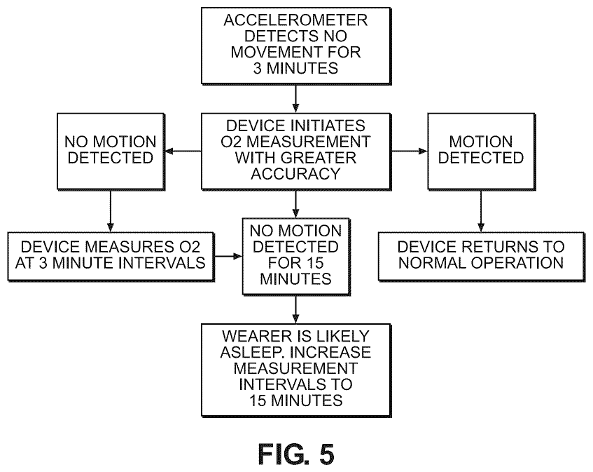

FIG. 5 is an exemplary flow chart illustrating the use of an accelerometer to detect preferred opportunities to take a wearer's measurement (SpO₂ readings are more accurate when the wearer is still) and to reduce the frequency of measurements when the wearer is asleep to preserve battery life. In this exemplary embodiment, when an accelerometer detects a wearer's lack of movement for 3 minutes, the device may take the wearer's SpO₂ measurement. Such an SpO₂ measurement will typically be more accurate when the wearer is still. The device may be configured to take measurements at, for example, 3-minute intervals as long as no movement is detected, thereby obtaining a series of relatively accurate SpO₂ measurements. If the device detects no movement for a period greater than 15 minutes, the device may be programmed to increase the interval of regular SpO₂ measurements to a larger interval, such as 15 minutes, to save on battery power because the user is likely asleep, reducing the risk of an overdose event.

All references, including publications, patent applications, and patents, cited herein are hereby incorporated by reference to the same extent as if each reference were individually and specifically indicated to be incorporated by reference and were set forth in its entirety herein.

Aspects of the present disclosure are described herein with reference to flowchart illustrations and/or block diagrams of methods, apparatuses (systems), and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable instruction execution apparatus, create a mechanism for implementing the functions/acts specified in the flowchart and/or block diagram block or blocks.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A wearable opioid overdose detection and alert system comprising:
a wearable band coupled to an electronic components housing configured to be worn on a user's wrist;
a plurality of blood oxygen concentration sensors configured to measure the blood oxygen concentration of a user wearing said system from a plurality of locations on a user's wrist; and
a communication module capable of communicating electronic information from a control module and to another electronic device;
said control module further comprising a processor and memory and provided in electronic communication with said plurality of blood oxygen concentration sensors and said communication module, the control module configured to:
determine a user's current blood oxygen concentration by averaging values measured by each blood oxygen concentration sensor;
record a user's baseline blood oxygen concentration; and
activate an alarm program when a user's blood oxygen concentration falls by a first set amount over a first set period of time or falls by a second set amount;
said alarm program configured to:
communicate a notification to the user of a health event;
activate a cancellable alarm for a second set period of time; and
communicate an emergency notification to an emergency contact via said communication module if said cancellable alarm is not cancelled within said second set period of time.

2. The wearable opioid overdose detection and alert system as recited in claim 1,
wherein said first set period of time is equal to or greater than 180 seconds.

3. The wearable opioid overdose detection and alert system as recited in claim 1,
wherein said second set period of time is equal to or less than 60 seconds.

4. The wearable opioid overdose detection and alert system as recited in claim 1,
wherein said first set amount is a 3-5% drop in the user's blood oxygen concentration.

5. The wearable opioid overdose detection and alert system as recited in claim 1,
wherein said second set amount is a greater than 5% drop in the user's blood oxygen concentration.

6. The wearable opioid overdose detection and alert system as recited in claim 1,
wherein said emergency notification includes location information supplied by a location module provided in electronic communication with said control module.

7. The wearable opioid overdose detection and alert system as recited in claim 6,
wherein said emergency notification further includes altitude information supplied by a barometer provided in electronic communication with said control module.

8. The wearable opioid overdose detection and alert system as recited in claim 7,
wherein said altitude information is displayed as an estimated floor.

9. The wearable opioid overdose detection and alert system as recited in claim 1, further comprising an accelerometer configured to detect when a user is not moving.

10. The wearable opioid overdose detection and alert system as recited in claim 9,
wherein said control module determines a user's current blood oxygen concentration when the user is not moving.

11. The wearable opioid overdose detection and alert system as recited in claim 10,
wherein said control module determines a user's current blood oxygen concentration at regular intervals when the user is not moving.

12. The wearable opioid overdose detection and alert system as recited in claim 1,
wherein said control module records a user's baseline blood oxygen concentration via an input received from the user.

13. The wearable opioid overdose detection and alert system as recited in claim 1,
wherein said control module records a user's baseline blood oxygen concentration by averaging a set of blood oxygen concentration measurements obtained over a set period of time initiated by the user.

14. The wearable opioid overdose detection and alert system as recited in claim 13,
wherein the set period of time for recording a user's blood oxygen concentration is greater than 180 seconds.

15. The wearable opioid overdose detection and alert system as recited in claim 1,
wherein said notification to the user comprises a sound generated by a speaker module provided in electronic communication with said control module.

16. The wearable opioid overdose detection and alert system as recited in claim 1,
wherein said notification to the user comprises a vibration generated by a vibration module provided in electronic communication with said control module.

17. The wearable opioid overdose detection and alert system as recited in claim 1,
wherein said cancellable alarm may be cancelled when the user presses and holds an analog button for a period greater than 3 seconds.

18. The wearable opioid overdose detection and alert system as recited in claim 1,
wherein said cancellable alarm may be cancelled when the user presses an analog button more than three times in a period less than five seconds.

19. The wearable opioid overdose detection and alert system as recited in claim 1,
wherein said communication module provided in wireless communication with a smartphone utilizes said smartphone to communicate said emergency notification.

20. A wearable opioid overdose detection and alert system comprising:
a wearable band coupled to an electronic components housing configured to be worn on a user's wrist;
a plurality of blood oxygen concentration sensors configured to measure the blood oxygen concentration of a user wearing said system from a plurality of locations on a user's wrist;
a location module capable of determining a user's location;
a barometer capable of determining atmospheric conditions corresponding to a user's altitude; and
a communication module capable of communicating electronic information from a control module and to another electronic device;
said control module further comprising a processor and memory and provided in electronic communication with said plurality of blood oxygen concentration sensors and said communication module, the control module configured to:
determine a user's current blood oxygen concentration by averaging values measured by each blood oxygen concentration sensor;
determine a user's baseline blood oxygen concentration by averaging measured blood oxygen concentration values over a period of time equal to or greater than 180 seconds at a time initiated by the user; and
activate an alarm program when a user's blood oxygen concentration falls by 3% or more over a 180 second period of time or falls by 5% or more for any period of time;
said alarm program configured to:
communicate a notification to the user of a health event;
activate a cancellable alarm for at least 45 seconds; and
communicate an emergency notification to an emergency contact via said communication module if said cancellable alarm is not cancelled within said cancellable alarm period, said emergency notification comprising location information provided by said location module and altitude information provided by said barometer displayed as a floor estimate.
